(19) United States
(12) Patent Application Publication
(10) Pub. No.: US 2012/0315301 A1
(43) Pub. Date: Dec. 13, 2012

(54) Preparation of vesicle-type carbon nanotubes

(75) Inventors: Mohsen Adeli, Khoramabad (IR); Masoumeh Bavadi, Khoramabad (IR); Masoumeh Ashiri, Khoramabad (IR); Masoumeh Hamid, Khoramabad (IR); Siamak Beyranvand, Khoramabad (IR)

(73) Assignee: Lorestan University

(21) Appl. No.: 13/067,546

(22) Filed: Jun. 8, 2011

(51) Int. Cl. A61K 9/00; A61K 47/30; A61K 9/127; B32B 9/00; D01F 9/12; A61K 47/04; B82Y 30/00

(52) U.S. Cl. 424/400; 423/447.2; 514/769; 428/402; 977/742

(57) ABSTRACT

A method to prepare new morphologies, especially vesicle-type, of carbon nanotubes (CNT) by supramolecular interactions between them and dendritic or linear-dendritic polymers and copolymers. Due to their water solubility, high functionality and unique properties, the prepared hybrid nanomaterials have excellent applicability in different fields especially in nanomedicine in comparison with usual CNTs.

### FIELD OF THE INVENTION

The invention relates to nanomaterials comprising carbon nanotubes that exhibit new morphologies, and/or solubility that make them particularly suitable for use as drug or gene delivery systems. The nanomaterials comprise carbon nanotubes (CNTs), dendritic or linear-dendritic polymers or copolymers, and/or metal nanoparticles.

### BACKGROUND

Due to their unique thermal, optical and chemical properties, carbon nanotubes, CNTs, have been intensively explored for biological and biomedical applications in the past few years [1-7]. They are able to transport therapeutic agents to tumors by “covalent” and “non-covalent” methods effectively. In the covalent method therapeutic agents are attached to their surface through chemical bonds while in the non-covalent method they are encapsulated in their cavity or loaded on their surface [8-12]. Their ability to cross the cell membranes besides the mentioned properties promises them as novel devices for cancer imaging and therapy [13-17]. However poor water solubility and low functionality are two major factors that limit the application of CNTs in the administration step and dominate their toxicity, biocompatibility, blood circulation time and biological properties [18-19]. Since safety and biocompatibility of CNTs is still under question and variety of research works are reported in this case [20-21], development of the new strategies to improve their water solubility and functionality is important [22]. However functionalization of CNTs changes their shape and conformation significantly [23]. While shape and size of nanomaterials—and especially CNTs—based drug delivery systems affect their toxicity efficiently [24, 25], investigation of the interrelation of functionalization, conformation and biocompatibility of CNTs can be important. Based on our research works, hydrophilic dendritic polymers not only rise the functionality, biocompatibility and water solubility of CNTs but also change their conformations dramatically [26].

A new method to improve both functionality and water solubility of CNT or CNT/metal hybrid nanomaterials using linear-dendritic copolymers without damaging their structure has been also reported. Linear-dendritic copolymers are hybrid nanomaterials consisting of linear and dendritic blocks [28-32]. Polyethylene glycol, PEG, is a well-studied and used polymer not only to synthesis variety of linear-dendritic copolymers but also to improve the process ability, water solubility and long blood circulation of CNTs through non-covalent interactions [33-38]. Hence supramolecular interactions between the PEG block of a linear-dendritic copolymers and CNTs leads to water soluble and high functional hybrid nanomaterials. PCA-PEG-PCA copolymers are biocompatible, water soluble and high functional ABA type linear-dendritic copolymers that has been synthesized and characterized and their potential as nanocarriers for drug delivery has been investigated previously [39-40].

### SUMMARY

One embodiment provides a nanomaterial comprising carbon nanotubes and having a new morphology, such as a morphology selected from liposome-like, vesicle-type, circle-type, spherical, etc., induced by supramolecular chemistry of the nanomaterial. In some embodiments, the nanomaterial exhibits a liposome or liposome-like morphology. In other embodiments, the nanomaterial exhibits a spherical or circle-type morphology. In some embodiments, the material has a core/shell structure based on carbon nanotubes. In some embodiments, the material is carbon nanotubes nanospheres.

Another embodiment provides a nanomaterial comprised of carbon nanotubes and polymers or copolymers, such as dendritic or linear-dendritic polymers or copolymers, and/or metal nanoparticles (“hybrid nanomaterials”). In some embodiments, the nanomaterial exhibits a vesicle-type morphology.

Another embodiment provides a nanomaterial comprised of carbon nanotubes and dendritic or linear-dendritic polymers or copolymers. In some embodiments the nanomaterial exhibits improved water solubility and/or functionality as compared to a material comprised only of carbon nanotubes. In some embodiments, the polymers or copolymers comprise functional groups, such as a large number of functional groups. In some embodiments, the functional groups are disposed on the surface of the nanomaterial. In other embodiments the functional groups are disposed within a cavity of the nanomaterial, such as within the cavity of a liposome-like structure.

Another embodiment provides carrier systems for transferring molecules or macromolecules (for example, drug or gene delivery systems) comprising a hybrid nanomaterial as described herein, e.g., comprising carbon nanotubes and dendritic or linear-dendritic polymers or copolymers and/or metal nanoparticles.

Another embodiment provides hybrid nanomaterials with supramolecular interactions and covalent linkages between carbon nanotubes (CNT) and dendritic or linear-dendritic polymers or copolymers. In some embodiments, the hybrid nanomaterials include a multi-walled carbon nanotube (MWCNT), single-walled carbon nanotube (SWCNT), opened carbon nanotube and/or a carbon nanotube decorated with metal nanoparticles. In some embodiments, the surfaces of the carbon nanotubes are decorated with nanoparticles or nanomaterials. In some embodiments, the carbon nanotubes are decorated by metal nanoparticles such as Fe, Mn, Ni, Co, Cr, Pt, and alloys thereof. In some embodiments, the metal nanoparticles decorated carbon nanotube are superparamagnetic. In some embodiments of the linear-dendritic polymers, the linear segment is a linear polymer or copolymer and the dendritic segment is a dendron, dendrimer or a hyperbranched polymer or their derivations. In some embodiments, the polymers or copolymers of the linear and dendritic segments are synthetic (such as polyethylene glycol, polyglycerol and polycitric acid) or natural macromolecules (such as starch and polylysine) or biomolecules (such as DNA, RNA and proteins).

Also provided are methods of making the nanomaterials described herein.

### BRIEF DESCRIPTION OF THE DRAWINGS

The present invention will be better understood in view of the following non-limiting figures, wherein:

FIG. 1 shows the preparation of hybrid nanomaterials through supramolecular interactions between carbon nanotubes and dendritic copolymers.

FIG. 2 (a) PG-PEG-PG, (b) PAMAM-PEG-PAMAM (second generation) and (c) PCA-PEG-PCA ABA type linear-dendritic copolymers, used to modify carbon nanotubes.

FIG. 3. Topographic AFM images of the coiled MWCNTs inside a bulk of PAMAM-PEG-PAMAM linear-dendritic copolymers (second generation).

FIG. 4. (a and b) TEM images of LLNs recorded using a waked electron beam. A coiled MWCNT forced by linear-dendritic copolymers form the skeletal backbone of LLNs. (c) The intensive electron beam of the TEM open the spherical LLNs, but PAMAM-PEG-PAMAM linear-dendritic copolymers are still associated onto the surface of MWCNT.

FIG. 5. (a) Topographic and (b) phase-contrast images of a PCA-PEG-PCA/MWCNTs LLNs. Topographic image show that LLNs are spherical. Two phases concerning the rigid carbon nanotubes and soft linear-dendritic copolymers can be seen in the phase-contrast image. (c) Aqueous solutions with a high concentration of PCA-PEG-PCA linear-dendritic copolymers and MWCNTs were led to big particles with a big cavity.

FIG. 6. (a) Sheet-like assemblies of PG-PEG-PG linear-dendritic copolymers wrap around MWCNTs to avoid their interaction with the water molecules, (b) SEM image of pristine MWCNTs and (c) SEM image of PG-PEG-PG/MWCNT LLNs.

FIG. 7. Topographic AFM images of PG-PEG-PG/MWCNT LLNs. A shell with 100 nm thickness can be seen around a rigid core in the left image.

FIG. 8. IR spectra of (a) DOX, (b) Opened CNTs, (c) PAMAM-PEG-PAMAM, (d) PAMAM-1 (e) PAMAM-PEG-PAMAM/MWCNT LLNs (f) PAMAM-1/MWCNT LLNs and (g) DOX/PAMAM-PEG-PAMAM/MWCNT.

FIG. 9. ¹H NMR spectra of a) PAMAM-PEG-PAMAM, b) PAMAM-PEG-PAMAM/MWCNT LLNs and c) DOX/PAMAM-PEG-PAMAM/MWCNTs.

FIG. 10. (a) Topographic AFM image of the modified PAMAM-PEG-PAMAM linear-dendritic copolymers (PAMAM-1) associated onto the surface of a MWCNT through π-π staking. (b) Profile of the highlighted domains in the image 10a.

FIG. 11. Two domains onto the carbon nanotubes; a domain concerning the linear PEG block and another related to the staked dendritic blocks.

FIG. 12. (a) Phase-contrast AFM image of the object showed in the FIG. 11 a. Dark parts display the defect sites on the side-wall and head of the carbon nanotube. These parts do not have an effective π-π interaction with PAMAM-1. (b) TEM image of a MWCNT bended by staked PAMAM-1.

FIG. 13. IR spectra of (a) DOX, (b) opened MWCNTs, (c) PG-PEG-PG, (d) PG-PEG-PG/MWCNT LLNs and (e) DOX/PG-PEG-PG/MWCNTs.

FIG. 14. ¹H NMR spectra of (a) PG-PEG-PG, (b) PG-PEG-PG/MWCNT LLNs, (c) DOX/PG-PEG-PG/MWCNT.

FIG. 15. TEM image of the MWCNTs, shielded by PG-PEG-PG linear-dendritic copolymers. Highlighted part show a defect site, where carboxyl functional groups are created, interacting with the hydroxyl functional groups of PG-PEG-PG linear-dendritic copolymers.

FIG. 16. DLS diagrams of (a) PAMAM-PEG-PAMAM, (b) PAMAM-PEG-PAMAM/MWCNT LLNs and (c) DOX/PAMAM-PEG-PAMAM/MWCNTs.

FIG. 17. DLS diagrams of (a) PG-PEG-PG (b) PG-PEG-PG/MWCNT LLNs and (c) DOX/PG-PEG-PG/MWCNTs.

FIG. 18. Raman spectra of a) PAMAM-PEG-PAMAM, b) PAMAM-PEG-PAMAM/MWCNT LLNs and c) DOX/PAMAM-PEG-PAMAM/MWCNTs.

FIG. 19. TGA analysis of (a) PAMAM-PEG-PAMAM, (b) PAMAM-PEG-PAMAM/MWCNT LLNs and (c) DOX/PAMAM-PEG-PAMAM/MWCNT. The result of dissociation of DOX molecules from the surface of MWCNTs and association of the PAMAM blocks of linear dendritic copolymer onto their surface is an exergonic process (The highlighted region in FIG. 19 c.

FIG. 20. TGA analysis of a) PG-PEG-PG, b) PG-PEG-PG/MWCNT LLNs and c) DOX/PG-PEG-PG/MWCNT.

FIG. 21. (a) Topographic and (b) the phase contrast AFM images of a PG-PEG-PG/MWCNT LLN containing encapsulated DOX molecules.

FIG. 22. Representation of the structure of liposome-like nanotubes (LLNs) and explanation of the role of carbon nanotube and dendritic polymer in this structure. Dendritic blocks (210) play the role of polar heads in liposome structure. Carbon nanotubes (220) play the role of lipidic bilayer in liposome structure

### DETAILED DESCRIPTION

Before explaining the disclosed embodiment of the present invention in detail it is to be understood that the invention is not limited in scope by the specific embodiment described herein, which are intended for the purpose of exemplification only.

The present invention relates to the preparation of novel hybrid nanomaterials comprised of carbon nanotubes (CNTs) and polymers or copolymers, or comprised of CNTs and metal nanoparticles, or comprised of CNTs, polymers or copolymers, and metal nanoparticles (collectively, “hybrid nanomaterials”). The polymers or copolymers may be dendritic or linear-dendritic (with linear segments and dendritic segments).

Supramolecular interactions between carbon nanotubes and linear-dendritic polymers or copolymers led to formation of liposome-like nanotube structures (LLNs). That is, the hybrid nanomaterials may form structures with liposome-like or vesicle-type morphologies, including spherical structures. The hybrid nanomaterials are useful, for example, as carrier systems for transferring molecules or macromolecules, such as drug or gene delivery systems.

As used herein, carbon nanotubes include multi-walled carbon nanotubes (MWCNTs), single-walled carbon nanotubes (SWCNTs), pristine carbon nanotubes, opened carbon nanotubes and metal nanoparticle decorated carbon nanotubes, and derivatives thereof.

As used herein, dendritic or linear-dendritic polymers and copolymers contain linear and dendritic segments. The linear and dendritic segments comprise linear polymers or copolymers, and dendron, dendrimer or a hyperbranched polymer and their derivations, respectively. In some embodiments of the linear-dendritic polymers, the linear segment is a linear polymer or copolymer and the dendritic segment is a dendron, dendrimer or a hyperbranched polymer or their derivations. In some embodiments, the polymers or copolymers are synthetic (such as polyethylene glycol, polyglycerol and polycitric acid) or natural macromolecules (such as starch and polylysine) or biomolecules (such as DNA, RNA and proteins). In some embodiments, the polymers or copolymers are hydrphillic.

Embodiments of the present invention provide a simple approach to obtain high functional and water soluble liposome like or vesicle-type hybrid nanomaterials, such as compared to a material comprised only of carbon nanotubes. For example, in some embodiments, the polymers or copolymers comprise functional groups, such as a large number of functional groups. In some embodiments, the functional groups are disposed on the surface of the nanomaterial. In other embodiments the functional groups are disposed within a cavity of the nanomaterial, such as within the cavity of a liposome-like structure.

In some embodiments, the invention provides carbon nanotubes with high functionality and/pr water solubility having morphologies of carbon nanotubes (CNTs) using dendritic or linear-dendritic polymers and copolymers.

In some embodiments, the surfaces of the carbon nanotubes are decorated with nanoparticles or nanomaterials. In some embodiments, the carbon nanotubes are decorated by metal nanoparticles such as Fe, Mn, Ni, Co, Cr, Pt, and alloys thereof. In some embodiments, the metal nanoparticles decorated carbon nanotube are superparamagnetic. By “decorated” means that nanoparticles or nanomaterials are deposited or attached onto the surface of the carbon nanotubes.

Conjugation of the paclitaxol molecules to the end functional groups of a hyperbranched poly(citric acid) grafted onto the surface of multi-walled carbon nanotubes (MWCNTs) leads to LLNs [27]. The main reason to form LLNs from MWCNT-graft-polycitric acid is non-covalent interactions between the functional groups of the grafted hyperbranched polymer and looping the carbon nanotubes to avoid interactions with water molecules.

According to exemplary implementations of the present invention for example, Fe₂O₃ nanoparticles, γ-Fe₂O₃NP, were deposited onto the surface of multi walled carbon nanotubes to obtain CNT/γ-Fe₂O₃NP hybrid nanomaterials. Non-covalent interactions between linear-dendritic copolymers and CNT/γ-Fe₂O₃NP hybrid nanomaterials led to the formation of a liposome-like or vesicle-type structure, making the hybrid nanomaterials useful as new drug delivery systems.

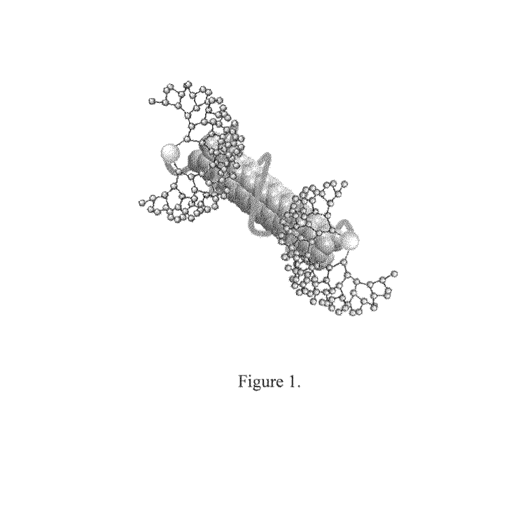

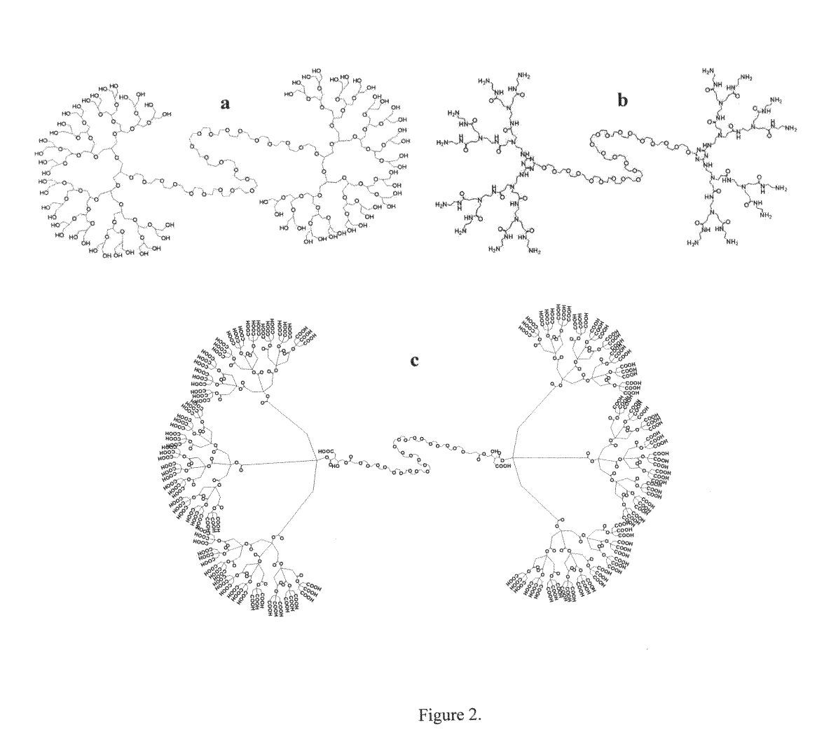

FIG. 1 is a schematic representation of the preparation of hybrid nanomaterials according to a particular embodiment of the present invention. As it is shown in FIG. 1, supramolecular interactions between carbon nanotubes surface and linear-dendritic copolymers lead to highly functional hybrid nanomaterials. While CNTs are usually insoluble in water due to their strong tendency to aggregate, most biological or biomedical applications require that the CNTs readily dissolve in aqueous media. In this embodiment three types of hydrophilic linear-dendritic copolymers including polyamidoamine-poly(ethylene glycol)-polyamidoamine (PAMAM-PEG-PAMAM), poly(citric acid)-poly(ethylene glycol)-poly(citric acid) (PCA-PEG-PCA) and polyglycerol-poly(ethylene glycol)-polyglycerol (PG-PEG-PG) ABA type linear-dendritic copolymers were synthesized and used to modify carbon nanotubes and solubilize them in water by supramolecular interactions (FIG. 2).

Simple mixing and sonicating of MWCNTs and linear-dendritic copolymers led to their aqueous solutions that were stable over several months.

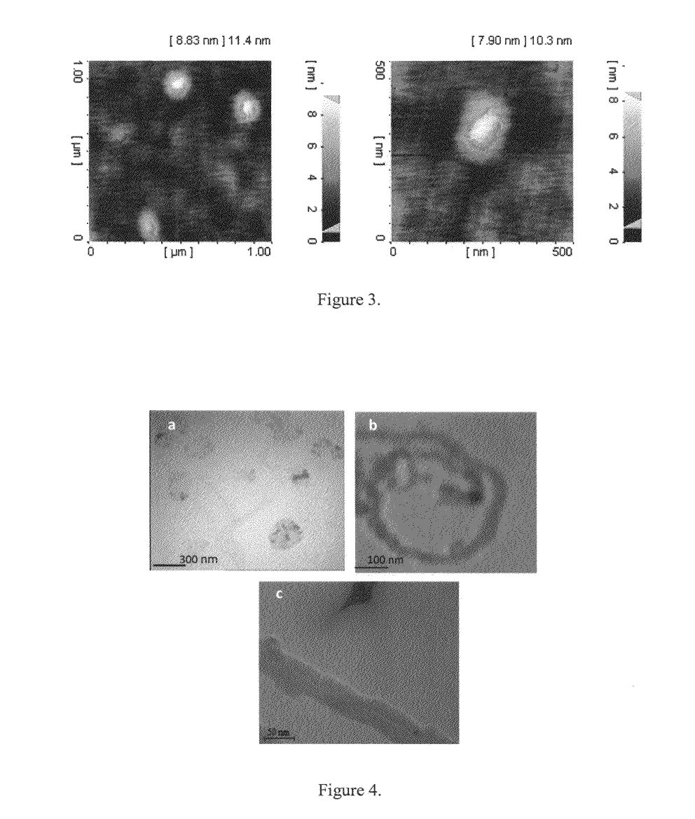

When linear-dendritic copolymers were used to modify CNTs, it was found that they changed their conformation from linear to packed state and led to LLNs. Another significant result was that, the geometry of LLNs depended on the type of dendritic blocks of linear-dendritic copolymers and their functional groups. FIG. 3 shows the AFM images of MWCNTs interacting with PAMAM-PEG-PAMAM linear-dendritic copolymers (PAMAM-PEG-PAMAM/MWCNTs). PAMAM-PEG-PAMAM/MWCNTs are shown as discrete LLNs in which a carbon nanotube is coiled inside a bulk of linear-dendritic copolymers. Diameter of LLNs is 200 nm and length of the coiled carbon nanotubes is around 630 nm. In the TEM image, recorded using a weak electron beam, LLNs are appeared as spherical objects with a 300 nm average size (FIG. 4 a). Dark objects in the LLNs are assigned to the coiled MWCNTs and other parts show the assembled PAMAM-PEG-PAMAM linear-dendritic copolymers (FIG. 4 b). Exposing this sample to the intensive electron beam of TEM opens LLNs but the linear-dendritic copolymers do not separate from the surface of the MWCNT, proving that there are strong interactions between them. Thickness of the assembled PAMAM-PEG-PAMAM linear-dendritic copolymers on the surface of MWCNT was easily found by calculation difference between the diameter of the primary MWCNT (20 nm) and PAMAM-PEG-PAMAM/MWCNTs (45 nm) (FIG. 4 c).

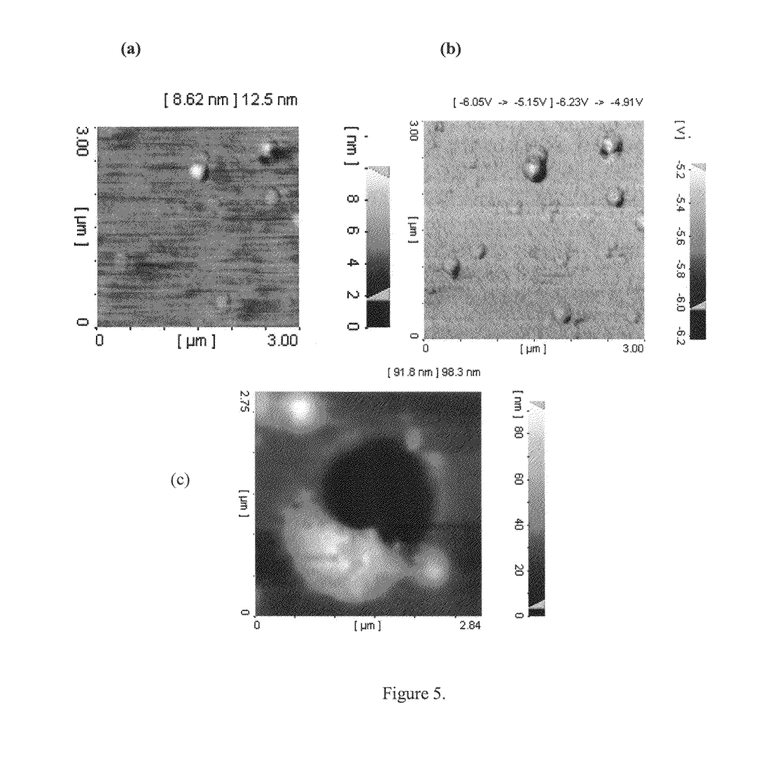

In spite of the PAMAM-PEG-PAMAM linear-dendritic copolymers, LLNs resulted from the non-covalent interactions between MWCNTs and PCA-PEG-PCA linear-dendritic copolymers (PCA-PEG-PCA/MWCNTs) were containing an empty cavity (FIG. 5). The average diameter and thickness of the shell of LLNs were 150-300 and 80 nm respectively and the average of volume of their cavity was 1.76×10⁶ nm³. Size of the PCA-PEG-PCA/MWCNTs LLNs was directly dependant on their concentration in the solution state so that in the high concentrations big particles with several micrometers diameter and an empty cavity were formed (FIG. 5c).

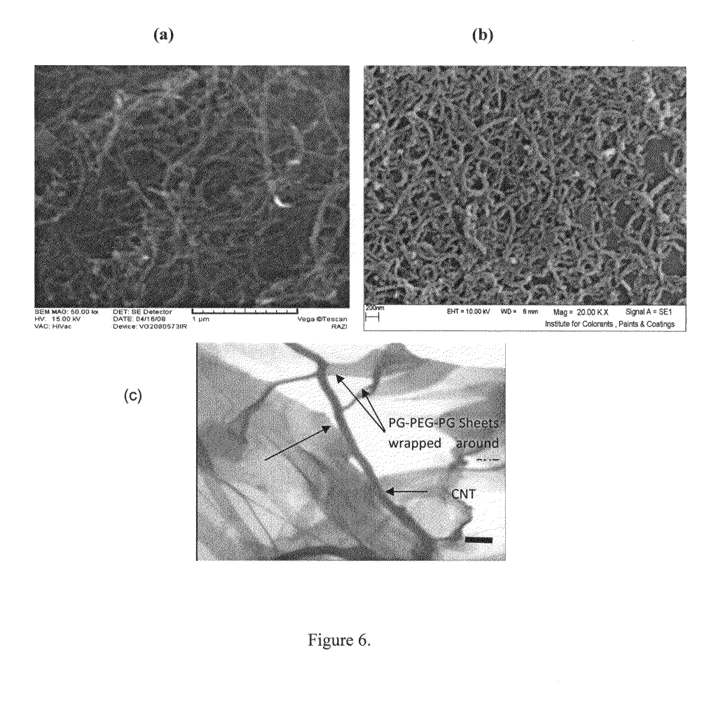

Comparison the SEM images of the acid treatment MWCNTs and PG-PEG-PG/MWCNT LLNs shows that carbon nanotubes are covered by linear-dendritic copolymers and they are not in their extended conformation (FIG. 6 a and b). The average thickness of the PG-PEG-PG/MWCNT LLNs is 50 nm. While diameter of the acid treatment MWCNTs is 20 nm, thickness of the polymeric shell assembled on their surface is 30 nm. FIG. 6c shows opening of the polymeric shell wrapped around a MWCNT, due to the impacting of the electron beam of TEM. As it can be seen, PG-PEG-PG linear-dendritic copolymers form a sheet-like assembly and then wrapped around MWCNTs. TEM images show that PG-PEG-PG linear-dendritic copolymers are wrapped around individual MWCNTs and observed objects in the SEM images are not containing bundles of MWCNTs.

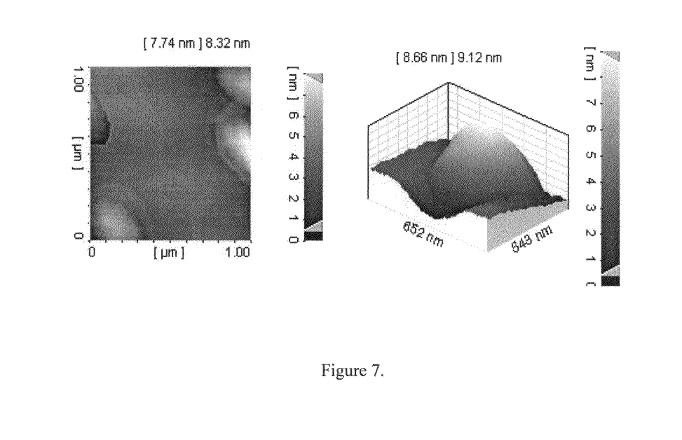

Interactions between the end functional groups of the sheet-like assemblies wrapped around the MWCNTs then lead to the LLNs (FIG. 7 a). PG-PEG-PG/MWCNT LLNs are not soft, like PAMAM-PEG-PAMAM/MWCNT LLNs, to see caged carbon nanotubes using AFM. MWCNTs are buried by PG-PEG-PG linear-dendritic copolymers rigidly so that they can not be recognized by AFM cantilever (FIG. 7 b).

Spectroscopy data show that the carbonyl functional groups of the dendritic blocks of PAMAM-PEG-PAMAM and PCA-PEG-PCA linear-dendritic copolymers dominate their non-covalent interactions with the surface of carbon nanotubes.

Two absorbance bands corresponded to the acidic and esteric carbonyl functional groups of PCA blocks of PCA-PEG-PCA linear-dendritic copolymers are exhibited at 1700 and 1740 cm⁻¹ respectively. In the IR spectra of PCA-PEG-PCA/MWCNT LLNs both absorbance bands are shifted toward lower frequencies proving that there is strong interactions between PCA blocks and MWCNTs. On the other hand intensity of the absorbance band of the esteric carbonyl functional groups is decreased significantly, due to the reduced polarity of these groups. Both observations confirm the electron transferring from MWCNT to the C═O bonds. Absorbance band of the aliphatic C—H bonds is shifted toward higher frequencies confirming that —CH₂—O— bonds of PEG are also interacting with MWCNTs.

Comparison of the ¹H NMR spectra of PCA-PEG-PCA linear-dendritic copolymers and PCA-PEG-PCA/MWCNT LLNs shows that peak surface area of protons of PEG and methylene groups of PCA blocks do not change upon non-covalent interactions with the surface of MWCNTs. Therefore it can be deduced that the etheric bonds of PEG and C—C bonds of PCA blocks are either equally interacting with the MWCNT or do not have a significant interaction with the surface of MWCNTs. Peak surface area of protons of both PEG and PCA blocks decreased significantly upon coordination of cisplatin (CDDP) molecules to the carboxyl functional groups of PCA-PEG-PCA linear-dendritic copolymers. This result prove that conjugation of CDDP molecules to linear-dendritic copolymers increase interactions between PEG and PCA blocks and MWCNT which is in agreement with the last result founded by the IR spectra.

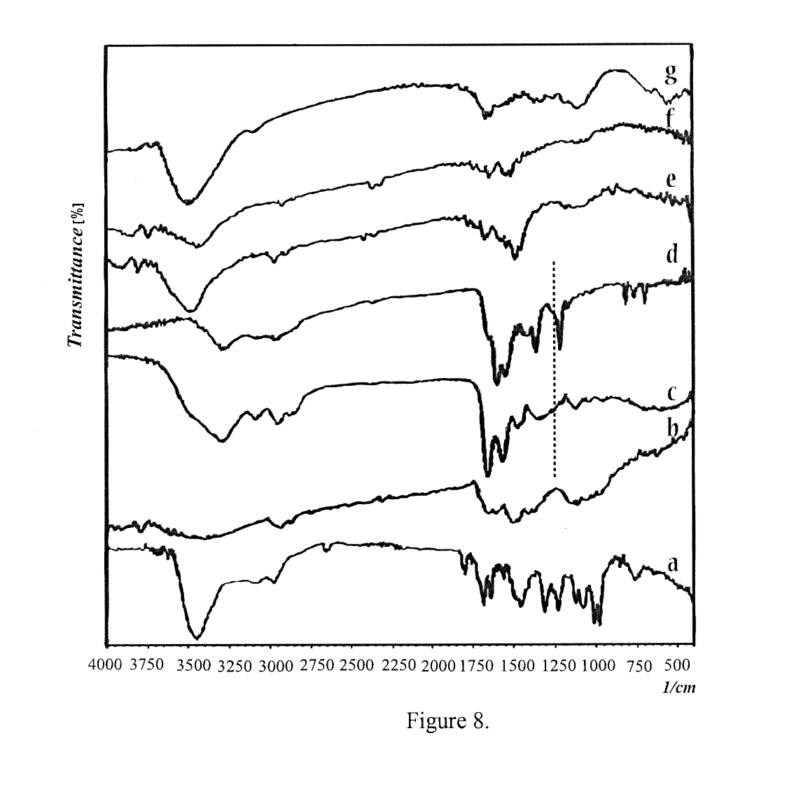

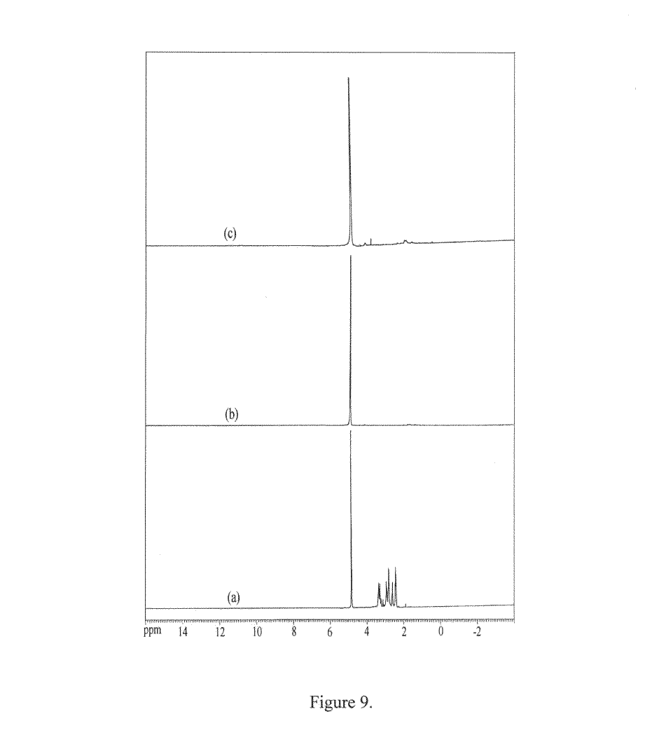

Intensity of the absorbance bands corresponded to the carbonyl functional groups of PAMAM-PEG-PAMAM linear-dendritic copolymer decrease intensively upon non-covalent interactions with MWCNTs confirming that the polarity of the carbonyl functional groups of PAMAM-PEG-PAMAM linear-dendritic copolymer diminish after staking on MWCNTs. This result can be assigned to the electron transferring from the π system of the MWCNT to the carbonyl functional groups of PAMAM-PEG-PAMAM linear-dendritic copolymer (FIG. 8).

Based on spectroscopy data it can be suggested that interactions between the π bonds of the carbonyl functional groups of linear-dendritic copolymers and π system of carbon nanotubes is the main driving force to stake linear-dendritic copolymers onto the surface of carbon nanotubes. In order to investigate this suggestion, 2-chloro-4,6-diphenyl-1,3,5 triazine was synthesized and conjugated to the amino functional groups of the second generation of PAMAM-PEG-PAMAM linear-dendritic copolymer and obtained copolymer (PAMAM-1) was interacted with the MWCNTs (Scheme 1).

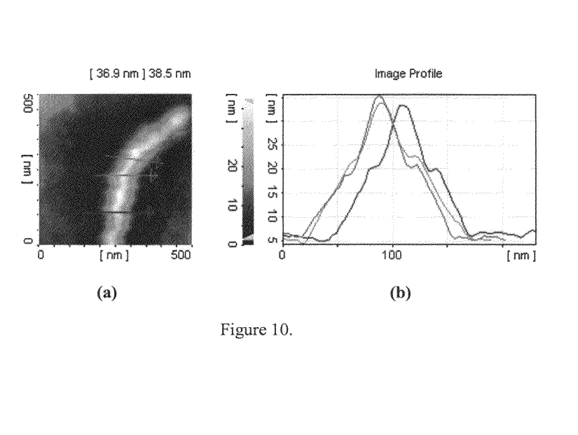

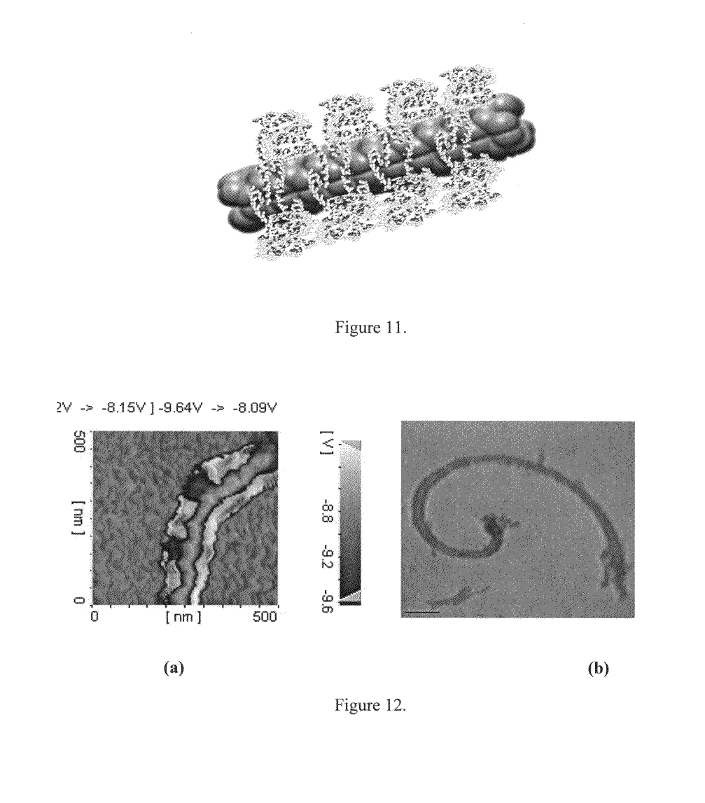

AFM images show that PAMAM-1 are staked onto the surface of carbon nanotube intensively (FIG. 10 a). Profile of the AFM images is including two parts (FIG. 10 b). The broad part which is 75 nm is assigned to a region of the surface of MWCNT which is covered by the staked dendritic blocks and narrow part is assigned to an area of the surface of MWCNT which is interacting with the linear poly(ethylene glycol) (FIG. 11). In the defect sites, produced during purification process in the both ends and sidewalls of carbon nanotubes, conjugated π system is loosened leading to diminished π-π staking interactions between the end aromatic groups of PAMAM-1 and surface of the carbon nanotube (FIG. 12). TEM images show that staked PAMAM-1 change the conformation of MWCNTs from extended (linear) toward closed (coiled) state.

PG-PEG-PG linear-dendritic copolymer does not have carbonyl functional groups or any functional group containing π bonds to interact with the conjugated π system of carbon nanotubes. However spectroscopy data, thermal analysis and visual observations show that they interact with the surface of MWCNTs strongly.

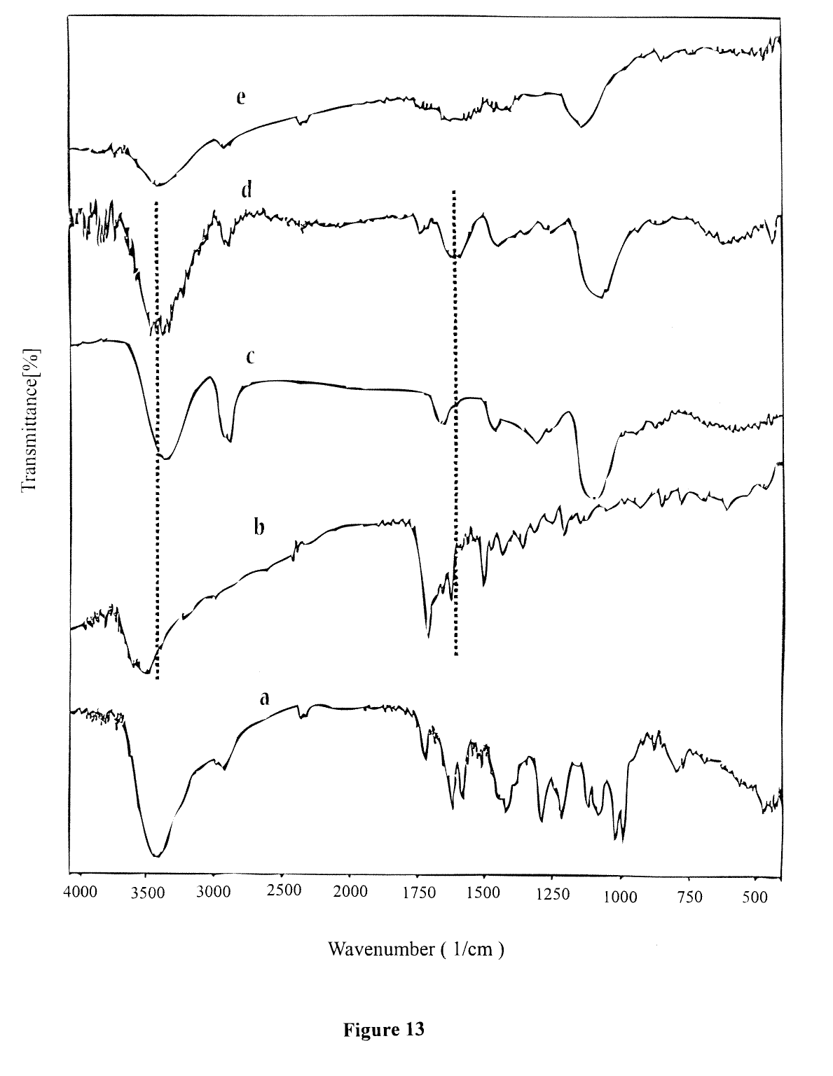

Carboxyl functional groups (—OH and carbonyl) of MWCNTs and hydroxyl functional groups of polyglycerol shift toward lower and higher frequencies respectively, proving that interactions between these functional groups have critical role in the interactions between MWCNTs and PG-PEG-PG linear-dendritic copolymers (FIG. 13).

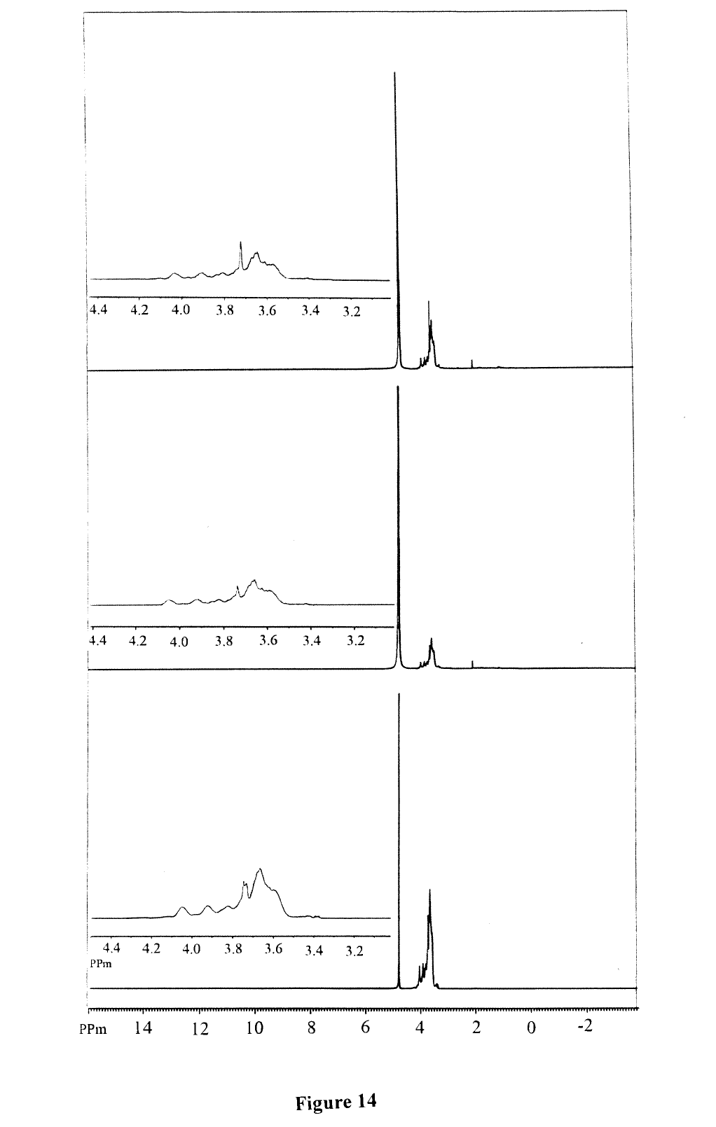

Interactions between MWCNTs and PG-PEG-PG linear-dendritic copolymer causes a broadening in the signals of both PEG and PG blocks. Based on this data PG and PEG blocks are interacting with the surface of MWCNTs surface equally. A reason for this observation is the similarity of the structures of PEG and PG blocks (FIG. 14).

Based on variety of data the main driving forces to form LLNs from PG-PEG-PG linear-dendritic copolymers and MWCNTs are as below:

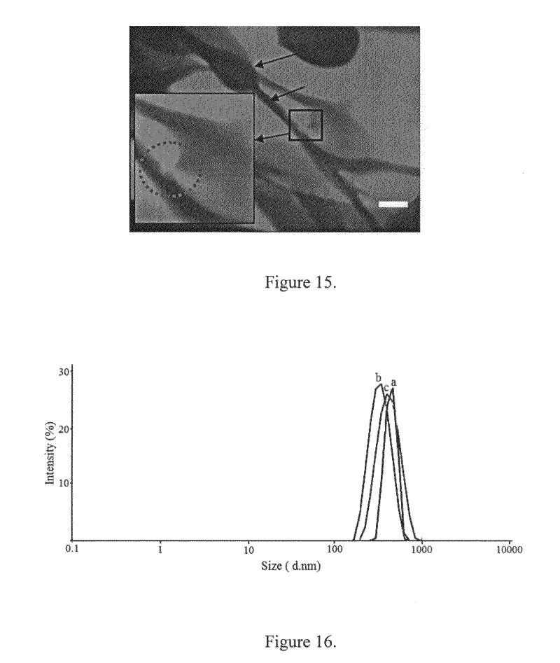

i) Interactions between hydroxyl functional groups of the PG-PEG-PG linear-dendritic copolymer and carboxyl functional groups in the defect sites and opened tips of the MWCNTs (FIG. 15).

ii) Avoiding interactions between water molecules and hydrophobic surface of the MWCNTs, which leads to increased entropy, by forming a shell around them.

DLS experiments show that the size of LLNs in the solution state is close to that measured by AFM in the solid state. Therefore they keep their geometry in the solution state, so that their inner space is still survived to encapsulate the therapeutic agents or other small molecules. To prove the efficacy of the LLNs as drug delivery systems, their ability to load doxorubicin (DOX) and cisplatin (CDDP) was investigated and then they were subjected to the endocytosis and release the drug inside the cancer cells (L929 and C26).

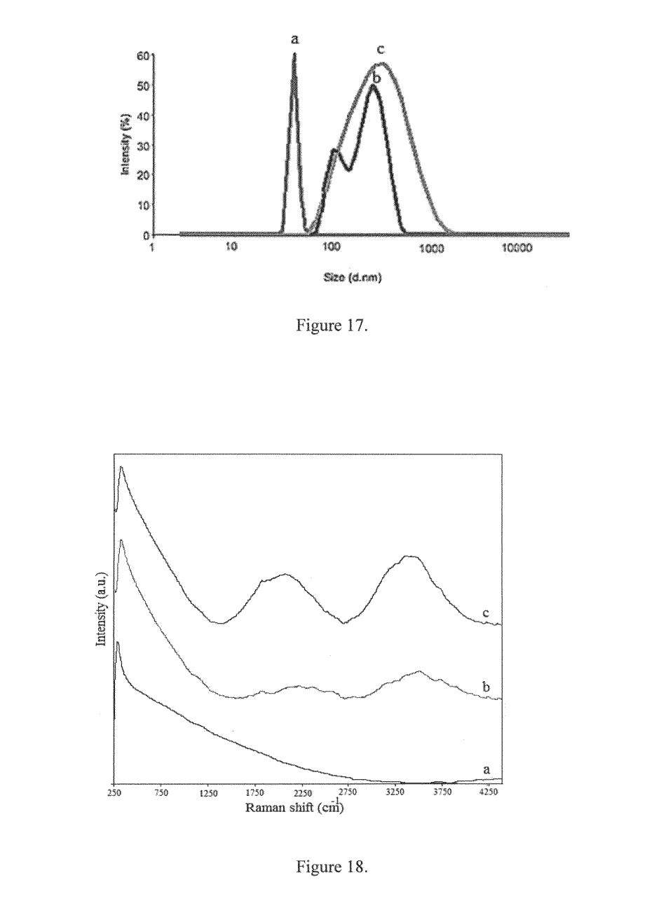

It was found that the encapsulation of DOX molecules by PG-PEG-PG/MWCNT and PAMAM-PEG-PAMAM/MWCNT LLNs increases their sizes. Because π-π staking of DOX molecules onto the surface of carbon nanotubes decrease interactions between linear-dendritic copolymers and the surface of carbon nanotubes, causing a less packed conformation for MWCNTs (FIGS. 16 and 17). Increasing of the peak surface area of the PEG block after adding a solution of DOX molecules to the aqueous solutions of the PG-PEG-PG/MWCNT LLNs prove loading of DOX molecules onto the surface of MWCNTs and therefore decreasing interactions between this surface and PEG block. Appearing of the signals of PAMAM-PEG-PAMAM linear-dendritic copolymer in the ¹H NMR spectra of DOX/PAMAM-PEG-PAMAM/MWCNT proves that non-covalent interactions between linear-dendritic copolymers and surface of the carbon nanotubes is interfered by loaded DOX molecules.

Due to the inter-connections of the dendritic blocks by CDDP molecules, size of the PCA-PEG-PCA/MWCNT LLNs decreases upon loading of CDDP molecules. A addition of the CDDP molecules to the PCA-PEG-PCA/MWCNT LLNs also causes disappearing of the absorbance band of the acidic carbonyl functional groups of PCA blocks in the IR spectra. These results show that CDDP molecules are conjugated to the linear-dendritic copolymers by coordinating to their carboxyl functional groups. Shifting of the absorbance bands of the esteric carbonyl functional groups toward lower frequencies can be assigned to their more effective interactions with carbon nanotubes after conjugation of CDDP molecules to their hydroxyl functional groups.

The Raman spectroscopy provides more interesting results concerning interactions between MWCNT and linear-dendritic copolymers and loading the drugs on them.

Raman spectra pattern of MWCNTs is loosed upon interactions with PG-PEG-PG and PAMAM-PEG-PAMAM linear-dendritic copolymers showing that the structural backbone of MWCNTs is deformed completely and they are not in their usual conformation (FIG. 18).

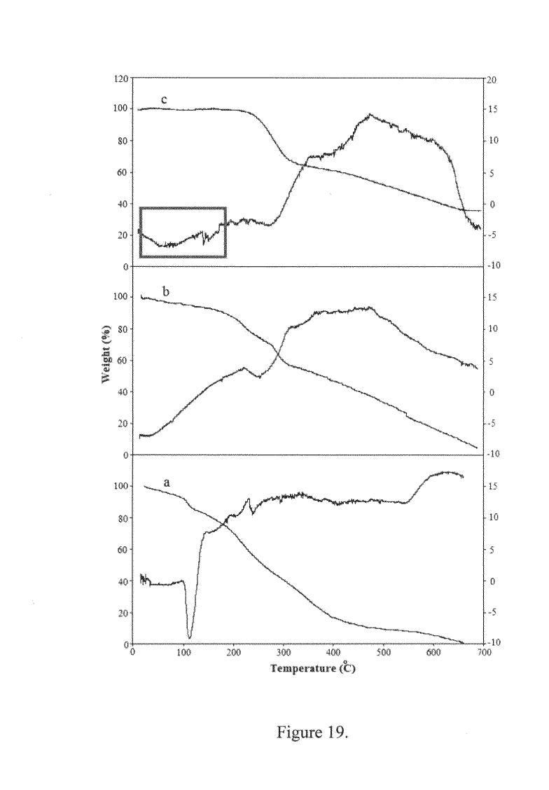

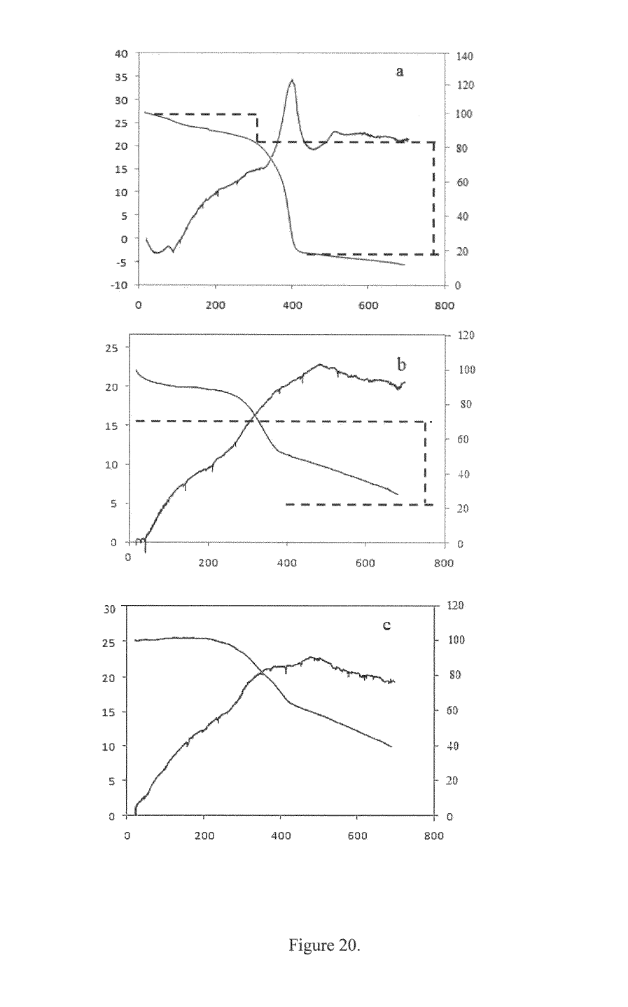

TGA-DTA analysis were also used to evaluate interactions between linear-dendritic copolymers and MWCNTs and encapsulation of DOX and CDDP molecules inside LLNs (FIGS. 19 and 20). While the DTA diagrams of both PAMAM-PEG-PAMAM and PG-PEG-PG linear-dendritic copolymers are endothermic between 0-130° C., these diagrams are exothermic in this region for PAMAM-PEG-PAMAM/MWCNTs and PG-PEG-PG/MWCNT LLNs. This result shows that non-covalent interactions between linear-dendritic copolymers and MWCNTs are exergonic and to disassociate them from the surface of carbon nanotubes, a considerable energy is needed. On the other hand, since the samples are heated in the solid state one of the possible driving forces for interactions between linear-dendritic copolymers and carbon nanotubes i.e avoiding interactions between water molecules and hydrophobic surface of carbon nanotubes, entropy factor, is not included here. There is a big difference between the DTA diagrams of DOX/PAMAM-PEG-PAMAM/MWCNTs and DOX/PG-PEG-PG/MWCNTs at 0-130° C. area so that it is endothermic for the first case while for the later is still endothermic. As it was proved, PAMAM-PEG-PAMAM linear-dendritic copolymers were able to interact with the surface of carbon nanotubes through π-πstaking. When temperature is raised, the DOX molecules dissociate from the surface of carbon nanotubes leading to considerable interactions between carbonyl functional groups of PAMAM-PEG-PAMAM linear-dendritic copolymer and the bear surface of carbon nanotubes. Although the dissociation of DOX molecules from the surface of MWCNTs is endothermic, exergonic interactions between a large numbers of carbonyl functional groups of PAMAM blocks with this surface lead to an exothermic process in summation. After this step, increasing the temperature causes dissociation of the staked PAMAM-PEG-PAMAM linear-dendritic copolymers from the surface of MWCNTs endothermically (FIG. 19 c).

In the case of PG-PEG-PG/MWCNT LLNs, the first step is similar to that for PAMAM-PEG-PAMAM/MWCNT LLNs i.e separation of the staked DOX molecules from the surface of MWCNTs is endothermic but the second step do not compensate it, due to the absence of π bonds in the PG-PEG-PG linear-dendritic copolymer, therefore the summation of this process is endothermic. Endothermic dissociation of the PG-PEG-PG linear-dendritic copolymer shell from the surface of MWCNTs then, increase the slope of the DTA diagram (FIG. 20 c).

The observed weight-loss region in the TGA diagrams of PAMAM-PEG-PAMAM/MWCNT and PG-PEG-PG/MWCNT LLNs at 70-150° C. disappeared upon encapsulation of DOX molecules. This weight-loss is assigned to the evaporation of encapsulated water molecules inside LLNs. After replacing the water molecules by DOX molecules this weight-loss also disappeared (FIGS. 19 and 20). However this weight-loss region cannot be seen in the TGA thermogram of PCA-PEG-PCA/MWCNTs LLNs containing CDDP molecules, confirming that CDDP molecules are conjugated to the carboxyl functional groups of PCA-PEG-PCA linear-dendritic molecules and they are not encapsulated inside LLNs.

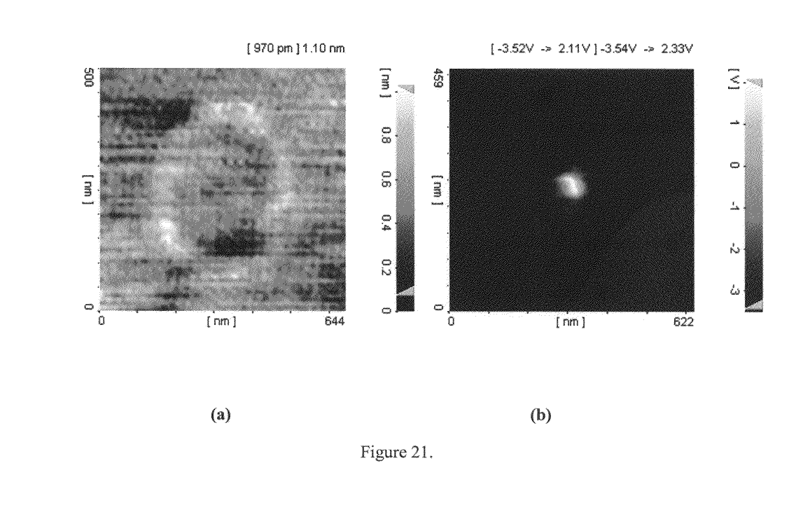

FIG. 21 display the AFM images of a PG-PEG-PG/MWCNT LLN containing encapsulated doxorubicin in its cavity. Diameter of LLN is 350 nm and length of the MWCNT should be one micrometer. Average of the thickness of the shell is 45 nm which is 15 nm more that the thickness of the primary MWCNTs. This difference is assigned to the PG-PEG-PG linear-dendritic copolymer assembled onto the surface of MWCNTs. Phase-contrast image show a different inner phase, proving the encapsulation of DOX molecules inside LLNs.

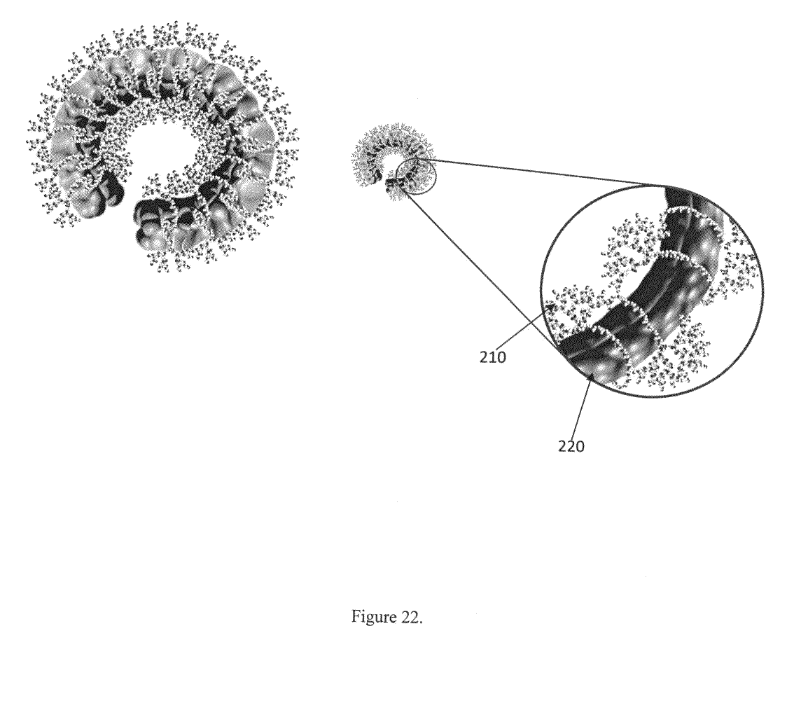

Based on the results of a variety of analysis and microscopy observations that are explained above, it is believed that the hybrid nanomaterials described herein exhibit a liposome-like structure comprised of carbon nanotubes and linear-dendritic copolymers, in which carbon nanotubes (220) and hydrophilic branches of linear-dendritic copolymers (210) play the roles of lipidic bi-layer structure and hydrophilic outside (polar heads) in liposome structure (FIG. 22).

Encapsulation of DOX molecules inside LLNs and their loading capacities were evaluated using UV-vis spectra and high performance chromatography.

According to HPLC experiments, loading capacities for PAMAM-PEG-PAMAM/MWCNT, PCA-PEG-PCA/MWCNT and PG-PEG-PG/MWCNT LLNs were: 3, 3.4, and 2.2 gram to one gram of LLNs respectively.

### EXAMPLES

### I. Materials

The multi wall carbon nanotubes MWCNT were prepared by chemical vapor deposition procedure in the presence of Co/Mo/MgO as catalyst at 900° C. Citric acid monohydrate (MW=210.14), poly ethylene glycol (MW=1000), cisplatin [Cis-Diamminedichloroplatinum (II), CDDP], [Fe (NO₃)₃.9H₂O] and HNO₃ were purchased from Merck. The cell lines (mouse tissue connective fibroblast adhesive cells (L929) were obtained from the National Cell Bank of Iran (NCBI) Pasteur institute, Tehran, Iran. 3-(4,5-dimethylthiazol-2-yl)-2,5-diphenyltetrazolium bromide (MTT) powder, Annexin-V FLUOS Staining Kit, was obtained from Sigma.

### II. Measurement Methods

Nuclear magnetic resonance (¹H NMR) spectra were recorded in D₂O solution on a Bruker DRX 400 (400 MHz) apparatus with the solvent proton signal for reference. Infrared spectroscopy (IR) measurements were performed using a Nicolet 320 FT-IR. Ultraviolet (UV) spectra were recorded on a shimadzu (1650 PC) scanning spectrophotometer. The particle size, polydispersity and zeta potential of materials were determined using Dynamic Light Scattering (DLS) (zetasizer ZS, Malvern Instruments). Thermogravimetric analysis (TGA) were carried out in a thermal analyzer (model: DSC 60, shimadzu, Japan) under dynamic atmosphere of an inert gas (i.e. N₂) at 30 ml/min (room temperature).

Morphology and size of materials were investigated using the Philips XL30 scanning electron microscope (SEM) with 12 and 15 A accelerating voltages.

Surface imaging studies were performed using atomic force microscopy (AFM) to estimate surface morphology and particle size distribution. The samples were imaged with the aid of Dualscope/Rasterscope C26, DME, Denmark, using DS 95-50-E scanner with vertical z-axis resolution of 0.1 nm. Raman spectra were obtained with an Almega Thermo Nicolet Dispersive Raman Spectrometer with second harmonic 532 nm of an Nd: YLF laser.

The Transmission electron microscopic (TEM) analyses were performed by a LEO 912AB electron microscope with accelerating voltage of 200 kV. The magnetic moment (M) of the hybrid nanomaterials were measured using Lake Shore model 7400 Vibrating Sample Magnetometer (VSM). Ultrasonic bath (Model: 5RS, 22 KHZ, Made in Italy) was used to disperse materials in solvents.

Simulation for NDDSs in the magnetic field was performed using Finite element method CONSOL multiphysics software.

### Example 1

### Opening of MWCNTs

The multi wall carbon nanotubes (MWCNTs) (2 g) were added to 30 ml of sulfuric and nitric acid mixture (3/1) in a reaction flask and refluxed for 24 h at 120° C. The mixture was cooled and diluted by distillated water and then it was filtrated. The product (MWCNT-COOH) was washed by distillated water and dried at 60° C. for 3 h by vacuum oven.

### Example 2

### Preparation of CNT/γ-Fe₂O₃NP Hybrid Materials

The multi wall carbon nanotubes (MWCNTs) (0.05 gr) were added into a solution of concentrated nitric acid containing iron nitrate (3 gr, 0.007 mmol) and refluxed for 4.5 h in an oil bath at 120° C. When the mixed solution was cooled to room temperature, the ammonia solution (2.5 wt %) was slowly added into the solution with vigorously stirring until the pH value reached 10. Then the solution was filtered with 0.65 mm filter membrane and washed with distilled water repeatedly. Later the product was dried overnight at 100° C. in an oven. The sample was then annealed at 250° C. for 1 h and 650° C. for 2 h in a steam of nitrogen.

### Example 3

### Preparation of PCA-PEG-PCA Copolymers

For synthesizing of PEG-g-PCA, (2gr) PEG (MW=1000, mol ratio=1/10) was added to a polymerization ampule equipped with magnetic stirrer and vacuum inlet. Monohydrate citric acid (7 gr) was also added to ampule and it was sealed under vacuum and then the mixture was stirred at 110° C. Produced water was removed by opening the vacuum inlet and temperature of reaction was slowly raised to 130° C. The vacuum inlet was closed and mixture was stirred at this temperature for 15 minutes. Then produced water was removed by opening the vacuum inlet and temperature of reaction was raised to 150° C. Polymerization was continued in this temperature under dynamic vacuum (open vacuum inlet) for 30 minutes in this temperature. The mixture was dissolved in tetrahydrofuran (THF) and was precipitated in diethyleter. Purified product was obtained as a viscous yellow compound in 80% yield [41].

### Example 4

### Preparation of CDDP-PCA-PEG-PCA-CDDP Prodrugs

CDDP (0.33 mmol) was suspended in 10 ml distilled water and mixed with silver nitrate ([AgNO₃]/[CDDP]=1) to form the aqueous complex. The solution was kept in dark at room temperature to appear AgCl precipitate. Then, the mixture was centrifuged at 7000 rpm for 20 min to remove the AgCl precipitates. Afterward, the supernatant was purified by passing through a 0.45 mm filter and PCA-PEG-PCA linear-dendritic copolymer (0.03 g) was added to above solution and gently stirred for 48 h at 37° C. to obtain the CDDP-PCA-PEG-PCA-CDDP conjugate.

### Example 5

### Preparation of PCA-PEG-PCA/CNT/γ-Fe₂O₃NP and CDDP/PCA-PEG-PCA/CNT/γ-Fe₂O₃NP Hybrid Nanomaterials

CNT/γ-Fe₂O₃NP (0.001 g) and PCA-PEG-PCA linear-dendritic copolymer (0.0024 g) (or CDDP-PCA-PEG-PCA-CDDP prodrug 0.00025 g) were mixed in distilled water (5 ml) and mixture was sonicated for 30 min at room temperature. Mixture was filtrated to obtain a clear brown solution.

### Example 6

### Preparation of PAMAM-PEG-PAMAM

Second generation of PAMAM-PEG-PAMAM linear-dendritic copolymer was prepared according to reported procedure in literature.

### Example 7

### Preparation of PAMAM-1

Ligand (2-chloro 4,6-di phenoxy-1,3,5-triazine) (compound a) that was synthesized according to reported procedure in literature was conjugated to the end amino functional groups of the second generation of PAMAM-PEG-PAMAM linear-dendritic copolymer (G₂). A solution of G₂ (2 g) in 5 mL of dry methanol was added to compound a (1.82 g) dissolved in 10 mL of dry dichloromethane, dropwise at 0° C. Mixture was stirred at room temperature for 1 h and refluxed for additional 12 h then it was cooled and filtered off. Solvent was evaporated and mixture was dissolved in 5 mL methanol and then product was precipitated in diethylether and purified compound was obtained as a dark brown viscose solid.

### Example 8

### Preparation of PAMAM-PEG-PAMAM/CNT LLNs

MWCNTs (2 mg) and PAMAM-PEG-PAMAM linear-dendritic copolymers (0.2 mg) were mixed in distilled water (5 ml) and mixture was sonicated for 30 min at room temperature. Mixture was filtered to obtain a clear black solution.

### Example 9

### Preparation of MWCNTs/PCA-PEG-PCA LLNs

MWCNTs/PCA-PEG-PCA LLNs were prepared according to our reported method in literature.

### Example 10

### Loading of CDDP Molecules by PCA-PEG-PCA/MWCNTs

CDDP molecules were loaded by MWCNTs/PCA-PEG-PCA LLNs according to our reported method in literature.

### Example 11

### Preparation of PG-PEG-PG/MWCNT_{S} LLNs

In this work, two molecular weights of PG-PEG-PG linear-dendritic copolymers were synthesized using different ratios of polyethyleneglycol (PEG) to glycidol (G) (1:10 and 1:20) and they were used to interact with MWCNTs non-covalently. PG-PEG-PG linear-dendritic copolymers (0.001 g) were dissolved in 5 ml distilled water and solution was added to MWCNTs (0.002 g).

### REFERENCES

1. Sun Y, Fu K, Lin Y, Huang W. Acc. Chem. Res. 2002; 35: 1096-1104.
2. Kharisov B, Kharissova O, Gutierrez H. L, Me´ndez U. O. Ind. Eng. Chem. Res. 2009; 48: 572-590.
3. Zhang P, Henthorn D. Langmuir 2009; 25(20): 12308-12314.
4. Cui H-F, Vashist S. K, Al-Rubeaan K, Luong J. H. T, Sheu F-S. Chem. Res. Toxicol. 2010; 23: 1131-1147.
5. Hu W, Peng Ch, Lv M, Li X, Zhang Y, ChenN, FanCh, HuangQ. ACSNANO. 2011, XXXX; XXX: ‘ 000-000’.
6. Ahmed M, Jiang X, DengZh, Narain R. Bioconjugate Chem. 2009; 20: 2017-2022.
7. Liu Zh, Sun X, Nakayama-Ratchford N, Dai H. ACS Nano, 2007; 1(1): 50-56.
8. Lou X, Daussin R, Pagnoulle Ch, Cuenot S, Detrembleur Ch, Duwez A-S, Bailly Ch, Je´rôme R. Chem. Mater. 2004; 16: 4005-4011.
9. Li R, Wu R, Zhao L, Wu M, Yang L, Zou H. ACS Nano 2010; 4(3):1399-1408.
10. Woo S, Lee Y, Sunkara V, Cheedarala R. K, Shin H. S, Choi H. Ch Park J. W. Langmuir 2007; 23: 11373-11376.
11. Khlobystov A, Britz D, Briggs A. Acc. Chem. Res. 2005; 38: 901-909.
12. Di Meo E. A, Di Crescenzo A, Demurtas D, Hubbell J, Velluto D, Fontana A, O'Neil C. Macromolecules. 2010; 43(7): 3429-3437.
13. Cheng J, Fernando K. A. S, Veca L. M, Sun Y-P, Lamond A. I, Lam Y. W, Cheng S. H. ACS Nano, 2008; 2(10): 2085-2094.
14. Prato M, Kostarelos K, Bianco A. Acc. Chem. Res. 2008; 41(1): 60-68. 2010; 9:793. b) Movia D, Del Canto E, Giordani S. J. Nature MaterialsKostarelos K.
15. (a) Phys. Chem. 2010; 114(43): 18407-18413. 2009; 4:627 (b) Pogodin S, Nature Nanotechnology Prato M. Bianco A, Kostarelos K,
16. (a) Baulin V. A. Acs Nano, 2010; 4(9):5293-5300.
17. (a) Kostarelos K, Bianco A, Prato M. Nature Nanotechnology 2010; 5:382. (b) Liu Q, Chen B, Wang Q, Shi X, Xiao Z, Lin J, Fang X. ACS Nano, 2009; 3 (9):2740-2750.
18. Dumortier H, Lacotte S, Pastorin G, Marega R, Wu W, Bonifazi D, Briand J-P, Prato M, Muller S, Bianco A. Nano Lett. 2006; 6(7):1522-1528.
19. (a) Bai Y, Zhang Y, Zhang J, Mu Q, Zhang W, Butch E R, Snyder S E, Yan B. Nature Nanotechnology 2010; 5:683-689. (b) Heister E, Lamprecht C, Neves V, Tîlmaciu C, Datas L, Flahaut E, Soula B, Hinterdorfer P, Coley H. M, Silva S. R. P, McFadden J. ACS Nano, 2010; 4(5):2615-2626.
20. Zhao Y, Xing G, Chai Z. Nature Nanotechnology 2008; 3:191.
21. (a) Ghafari P, St-Denis C H, Power M E, Jin X, Tsou V, Mandal H S, Bols N C, Tang X. Nature Nanotechnology 2008; 3: 347. (b) Schipper M K, Nakayama-Ratchford N, Davis C R, Kam N W S, Chu P, Liu Z, Sun X, Dai H, Gambhir S S. Nature Nanotechnology 2008; 3:216.
22. Buffa F, Hu H, Resasco D. Macromolecules. 2005; 38(20): 8258-8263.
23. Adeli M, Mirab N, Zabihi F. Nanotechnology 2009; 20: 485603 (10 pp).
24. Kostarelos K. Nature Biotechnology 2008; 26(7):774-776.
25. Tummala N. R, Morrow B. H, Resasco D. E, Striolo A. ACS Nano 2010; 4 (12): 7193.
26. Adeli M, Mirab N, Alavidjeh M S, Sobhani Z, Atyabi F. Polymer 2009; 50:3528.
27. (a) Adeli M, Hakimpoor F, Ashiria M, Bavadia M, Kabiri R. Soft Matter 2011; 7: 4062. (b) Sohhani Z, Adeli M, Dinarvand R, Ghahremani M, Atyabi F. Journal of Nanomedicine 2011; 6: 705.
28. (a) A. Simonyan A, Gitsov I. Langmuir 2008; 24:11431. (b) Adeli M, Zarnegar Z, Dadkhah A, Hossieni R, Salimi F, Kanani A. Journal of Applied Polymer Science 2007; 104:267.
29. Adeli M, Haag R. Journal of Polymer Science Part A Polymer Chemistry 2006; 44:5740.
30. Namazi H, Adeli M. Polymer 2005; 46:10788.
31. Namazi H, Adeli M. Journal of Polymer Science Part A Polymer Chemistry 2005; 43:28.
32. Tavakoli Naeini A, Adeli M, Vossoughi M. European Polymer Journal 2010; 46:165.
33. Adeli M, Zarnegar Z. Journal of Applied Polymer Science 2009; 113:2072
34. Yang K, Wan J, Zhang Sh Zhang Y, Lee Sh-T Liu Zh. Acsnano. 2011; 5(1): 516-522.
35. Li W-S, Aida T. Chem. Rev. 2009; 109: 6047-6076.
36. (a) Liu Z, Tabakman S M, Chen Z, Dai H. Nature Protocols 2009; 4(9):1372. (b) Berlin J. M. Leonard A. D, Pham T. T, Sano D, Marcano D. C., Yan S, Fiorentino S, Milas Z. L, Kosynkin D. V, Price B. K, Lucente-Schultz R. M, Wen X, Raso M. G, Craig S. L, Tran H. T, Myers J. N, Tour J. M. Acsnano. 2010; 4(8): 4621-4636.
37. Welsher K, Liu Z, Sherlock S P, Robinson J T, Chen Z, Daranciang D, et al. Nature Nanotechnology 2009; 4:773.
38. Namazi H, Adeli M. Biomaterials 2005; 26:1175.
39. Namazi H, Adeli M. European Polymer Journal 2003; 39:1491.
40. Namazi H, Adeli M, Zarnegar Z, Jafari S, Dadkhah A, Shukla A. Colloid and Polymer Science 2007; 285:1527.
41. Tavakoli Naeini A, Adeli M, Vossoughi M. Nanomedicine 2010; 6; 556.

What is claimed is:

1. A nanomaterial comprising carbon nanotubes and having a morphology selected from liposome-like, vesicle-type, circle-type, and spherical.

2. The nanomaterial of claim 1, comprised of carbon nanotubes and dendritic or linear-dendritic polymers or copolymers, and/or metal nanoparticles.

3. The nanomaterial of claim 2, comprised of carbon nanotubes and dendritic or linear-dendritic polymers or copolymers.

4. The nanomaterial of claim 3, wherein the polymer or copolymer comprises functional groups.

5. The nanomaterial of claim 4, wherein the functional groups are disposed on the surface of the nanomaterial.

6. The nanomaterial of claim 5, wherein the functional groups are disposed within a cavity of the nanomaterial.

7. The nanomaterial of claim 3, wherein the nanomaterial is soluble in water.

8. The nanomaterial of claim 9, wherein the carbon nanotubes are selected from the group consisting of multi-walled carbon nanotubes (MWCNTs), single-walled carbon nanotubes (SWCNTs), opened carbon nanotubes, and combinations thereof.

9. The nanomaterial of claim 2, wherein the carbon nanotubes are decorated with metal nanoparticles.

10. The nanomaterial of claim 9, wherein the metal nanoparticles are selected from the group consisting of Fe, Mn, Ni, Co, Cr, Pt, and alloys thereof, and combinations thereof.

11. The nanomaterial of claim 10, wherein the metal nanoparticles-decorated carbon nanotubes are superparamagnetic.

12. The nanomaterial of claim 2, wherein the polymer or copolymer is a linear-dendritic polymer or copolymer.

13. The nanomaterial of claim 12, wherein the linear-dendritic polymer or copolymer comprises a dendritic segment that is a dendron, dendrimer, hyperbranched polymer, or derivative thereof.

14. The nanomaterial of claim 2, wherein the polymer or copolymer is selected from synthetic polymers, natural macromolecules and biomolecules.

15. The nanomaterial of claim 2, having a liposome or liposome-like morphology.

16. The nanomaterial of claim 2, having a spherical or circle-type morphology.

17. The nanomaterial of claim 2, having a core/shell structure.

18. The nanomaterial of claim 2, in the form of carbon nanotubes nanospheres.

19. A carrier system for transferring molecules or macromolecules comprising a nanomaterial according to claim 1.

20. A method of making a nanomaterials according to claim 1 comprising mixing carbon nanotubes and linear-dendritic copolymers.
